(19) United States
(12) Patent Application Publication
(10) Pub. No.: US 2008/0269633 A1
(43) Pub. Date: Oct. 30, 2008

(54) Sonification of Level of Consciousness of a Patient

(75) Inventor: Marcus Watson, Queensland (AU)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: THE UNIVERSITY OF QUEENSLAND, Queensland (AU)

(21) Appl. No.: 12/158,674

(22) PCT Filed: Dec. 22, 2006
(86) PCT No.: PCT/AU2006/002009

§ 371 Date: Jun. 20, 2008

(30) Foreign Application Priority Data
Dec. 23, 2005 (AU) 2005907274

(51) Int. Cl. A61B 5/0476

(52) U.S. Cl. 600/544

(57) ABSTRACT

The level of consciousness of a patient (i.e. the hypnotic state and/or the level of anaesthesia) is represented aurally. A measure of the patient's level of consciousness, such as the BIS index value, is obtained, and an audio signal is synthesized from that measure, and then output through a speaker. Both the volume and pitch of the audio signal may vary according to the BIS value being represented, so that a clinician can obtain an indication of the level of consciousness of the patient simply by listening to the sonification of the BIS value. The audio signal may comprise first and second audio components, with the first component representing the previous measure and the second component representing the current measure. The amplitude or pitch of the audio signal may be modulated to represent analgesia or paralysis of the patient.

### FIELD OF THE INVENTION

This invention relates generally to the monitoring of the level of consciousness of a patient. In particular, the invention is directed to method and apparatus for sonification of a signal representing the level of consciousness of a patient, so that the level of consciousness can be monitored aurally by a clinician.

### BACKGROUND OF THE INVENTION

[Mere reference herein to prior art does not constitute an admission that such prior art constitutes admissible prior art, or common general knowledge in the art, in any particular country.]

Patients undergoing surgery or receiving other forms of intense therapy require an adequate level of hypnosis or anaesthesia to protect them from stress, awareness and recall of the traumatic experiences associated with the procedure. Clinicians such as anaesthetists must continually monitor and manage the hypnotic state and/or level of anaesthesia of patients during such procedures, or otherwise when administering anaesthetics and sedatives. If a patient's level of consciousness is too high, there is a risk that the patient may regain consciousness or have memory of the traumatic events of the procedure.

In addition, when a patient is recovering from a procedure, it is necessary to monitor their level of consciousness since a rapid “awakening” can be detrimental, while a slow awakening can result in an unnecessary over-administration of anaesthetic drugs.

It is also often desired to monitor a patient's level of brain activity when performing cardiopulmonary resuscitation (CPR) on a patient, as a primary purpose of CPR is to maintain the patient's brain function. In these situations, there can be difficulty in monitoring the level of consciousness of the patient, at least not without distracting the clinician or paramedic from other from functions.

Throughout this specification, the term “level of consciousness” of a patient shall mean the hypnotic state and/or the level of anaesthesia of the patient.

Unless a contrary intention applies, references herein to “the level of hypnosis” and “the hypnotic state” etc of a patient will be understood to mean the level of impairment of the patient's consciousness and memory, e.g. the state of being unconscious or subconscious. Similarly, unless a contrary intention applies, references herein to “the level of anaesthesia” of a patient will be understood to mean the impairment, or lack, of awareness of the patient, or their oblivion to external stimuli.

At low levels of hypnosis, an indirect assessment of the patient's hypnotic state can be obtained by observing physical signs and the patient's responsiveness, for example to voice and touch. However, assessment techniques such as these have significant limitations as they cannot be used in situations where the patient is unable to provide a response. Even in situations where these techniques can be used, the stimulation caused by the assessment itself may arouse the patient. In any event, these approaches provide only a subjective and instantaneous assessment of the patient's hypnotic state.

In order to overcome these limitations, attempts have been made to develop means for obtaining objective measurements of hypnotic state that can be acquired continuously and without disturbing the patient. One useful measure which is used for this purpose is the surface electroencephalogram (EEG). EEGs are complex physiological waveform signals which represent the sum of all brain activity produced by the cerebral cortex of a patient.

It is generally accepted that a patient's EEG generally changes from a low amplitude, high-frequency signal whilst the patient is conscious and alert, to a large amplitude, low frequency signal when the patient is deeply anaesthetised. Reported EEG representations of various levels of hypnosis are shown in FIGS. 1A to 1D. EEGs can also be used to provide an indication as to the extent of patient's lack of muscle activity (paralysis) and absence of pain (analgesia).

In order to provide clinicians with information regarding a patient's hypnotic state, various indices have been developed to provide a quantitative measure of the patient's hypnotic state based on EEG measurements at a given time. One such index is the Bispectral Index (BIS). A patient's BIS reading is a number between 0 and 100 indicating the patient's level of hypnosis. A BIS value near 100 indicates that the patient is in an “awake” clinical state, whilst a BIS value below approximately 10 denotes a disappearance of measured brain activity leading to an isoelectric EEG. Thus, low BIS values such as this indicate very deep hypnosis. The BIS index can also be used to provide an indication of a patient's level of paralysis and/or analgesia.

Measures of a patient's hypnotic state based on BIS values or similar indices are currently represented to clinicians using numerical and graphical visual displays. A significant disadvantage of such numerical and graphical displays is that the utility of the display, and hence the value of the information represented thereon, is dependent upon the clinicians' ability to regularly monitor the presented information visually whilst also attending to the needs of the patient. In some situations it is difficult or impossible for the clinician to monitor such visual displays adequately. This can lead to a possibility that the clinician may fail to notice important changes in the patient's level of consciousness, and in some extreme circumstances this may lead to the patient regaining appreciable levels of awareness or recall of an invasive medical procedure.

A system has been proposed to emit an audible alarm if the patient's level of consciousness falls outside a predetermined range. However, such an alarm type system does not does not provide continuous information regarding trends in the patient's level of consciousness.

The term “sonification” is generally used to describe the creation of an audio signal which is dependent upon the measured value of a parameter which is to be represented aurally. International patent application WO 03/017838 discloses sonification of respiratory behaviour. That method uses tones of different pitch to represent different levels of measured respiratory parameters, e.g. respiratory flow and carbon dioxide concentrations.

U.S. Pat. No. 6,947,780 discloses method and apparatus for sonification of physiological data, and in particular blood oximeter readings. An audio signal is generated at each pulse, dependent upon the measured oximeter reading. If the measurement corresponds to one of a plurality of pre-determined transition points in a range, a tone of a respective frequency is generated. For readings which fall between a pair of the transition points, a dual tone signal is generated. The dual tone signal comprises two frequencies, each of which has its amplitude modified by a respective factor which depends upon the proximity of the reading to the pair of transition points. Although no evaluation data is presented for this system of sonification, it seems likely that clinicians would find it difficult to distinguish between tones of closely spaced frequencies and between different amplitudes, particularly at typical pulse rates.

Although the concept of sonification is known, at present there are no methods that guarantee a successful sonification. This is evident in the very small number of successful sonifications used to date.

Sonification is more easily applied to represent physiological behaviour or parameters which can be easily measured. Parameters such as oxygen saturation or heart rate, used in the pulse oximetry sonification, have normal ranges that clinicians' would expect these parameters to be in. In the case of level of consciousness, in the past there has not be a widely accepted and easily measured quantitative measure of the patient's hypnotic state which is particularly suitable for sonification. Moreover, the level of consciousness will be dependent upon the particular moment in time. Level of consciousness has normally been represented with a visual graph augmented with auditory alarms. Studies have shown that many auditory alarms are often ignored, or considered to be a nuisance.

Since several physiological parameters may be displayed and monitored aurally during a surgical procedure, it is crucial that the different audio outputs be easily distinguishable. Each sonification must be designed to effectively convey the data it represents against background sounds. In some cases, the fact that other sounds provide important information, such as conversation, auditory alarms or other sonifications, means that the design of new sonifications to map data to sound is a very difficult task.

At first glance, sonification of level of consciousness does not appear to be particularly advantageous. During a surgical procedure, it desirable that the patient is unaware of what is happening while a surgeon is operating. This is normally indicated by a middle range on a consciousness scale. The clinician only needs consciousness information if the patient moves outside of the selected range. Auditory alarms have therefore been used to indicate when the patient moves outside of the set boundaries. Since sonifications provide continuous sound to represent data, the use of a sonification to represent a normal state would normally add extra noise and compete with other sonifications, e.g. pulse oximetry for attention. Since in most procedures, the patient will spend the majority of the time in an unconscious state, a sonification that provides continuous feedback will cause undue distraction to the operating theatre staff.

For the foregoing reasons, sonification has not hitherto been adopted for displaying a patient's level of consciousness.

However, when the surgical procedure has finished and the patient is emerging from unconsciousness, it is important to have a steady climb in the depth of conscious measure. At this point it is would be desirable to have information about the rate of change to provide confirmation to the clinician that the patient is recovering and also information about the rate at which awareness is returning to the patient.

It is an object of the present invention to provide method and apparatus for sonification of a signal representing the level of consciousness of a patient, so that the level of consciousness can be monitored aurally.

### SUMMARY OF THE INVENTION

In one broad form, the invention provides a method of indicating level of consciousness of a patient aurally, comprising the steps of

(a) obtaining a measure representative of the patient's level of consciousness, which is typically the patient's BIS value,

(b) synthesising an audio signal from the measure, and

(c) outputting the audio signal.

Steps (a) to (c) are repeated at regular intervals of time.

At least one audible characteristic of the audio signal is dependent upon the patient's level of consciousness. In this manner, a clinician can obtain an indication of the level of consciousness of the patient simply by listening to the sonification of the BIS value, i.e. without requiring the clinician to interrupt other tasks that they are performing in order to review the information on a visual display, as has previously been required.

The audible characteristic of the output audio signal that represents information about the patient's level of consciousness may include, but is not limited to, amplitude (i.e. loudness or volume), frequency (pitch), signal duration, sound sequences, sequences of pitch repetitions and/or pitch variations and combinations thereof, and effects such as vibrato, tremolo, crescendos, diminuendos, echoes and the like. Typically, the frequency (pitch) and/or amplitude (volume) of the audio signal varies with the BIS value. The audio signal may also comprise a combination of two or more frequencies (pitches) in which case the timbre of the audio signal created by the combination of the two or more frequencies comprises another audible characteristic.

Where the BIS Index is used as the measure of the patient's level of consciousness, the information presented aurally to the clinician by the audio signal may indicate the patient's current BIS value, or the range of BIS values into which the patient's hypnotic state currently falls. The invention is not limited to operation with the BIS Index, and may operate with any similar or analogous measure of the patient's level of consciousness, whether based on EEG measurements or on some other physiological parameter.

A clinician may define a desired level of consciousness for the patient. Where the invention uses the BIS Index, the desired level of consciousness may be specified by a range of BIS values. Typically, the desired hypnotic state for a patient under an appropriate degree of anaesthesia for a medical procedure will be between BIS values of about 45 and about 75.

Preferably, when the patient's level of consciousness falls within the desired range, the amplitude of the synthesised audio signal is low so as to be virtually inaudible. However, when the patient's level of consciousness changes so that it falls outside the defined desired range, the amplitude of the synthesised audio signal audibly increases to alert the clinician to the change.

The patient's BIS measure may stray above or below the desired BIS value range. In one embodiment of the invention, the amplitude of the audio signal increases according to the extent to which the BIS value is outside the predetermined range (i.e. irrespective of whether it is above or below in the predetermined range). The volume of the audio signal thereby indicates to the clinician the extent to which the patient's level of consciousness has strayed from the desired range. Other sound parameters (for example frequency or timbre) might alternatively be used to indicate that extent to which the patient's level of consciousness has strayed outside the desired range.

In the embodiments where amplitude is used to indicate the extent to which the BIS value deviates from the predetermined range, the change in loudness may depend on the amount of deviation according to any linear, nonlinear, stepped or other relationship, but preferably an equi-loudness relationship. By way of example only, if an equi-loudness relationship is used, the increase in volume apparent to the clinician when the patient's BIS value strays from a value of 75 to 76 will be the same as the apparent volume increase caused by a change from 74 to 75.

In order to indicate to the clinician whether the patient's level of consciousness has strayed above or below the desired range, the frequency of the audio signal may be varied accordingly. For example, if the patient's level of consciousness strays above the desired range, the audio signal will have a relatively high audible frequency (i.e. a relatively high pitch). Conversely, if the patient's level of consciousness strays below the desired range, then the audio signal will have a relatively low pitch.

Advantageously, the audio signal is modified in accordance with the patient's state of analgesia and/or paralysis. The modified audio signal thereby provides the clinician with an aural indication of the level of pain being experienced by the patient, in addition to the level of consciousness. In one embodiment, if the patient begins experiencing a level of pain that is higher than that desired by the clinician, this may be indicated to the clinician by a tremolo (i.e. a rapid rise and fall in volume) in the audio signal. Similarly, it is envisaged that if the patient's level of paralysis varies from that which is desired by the clinician, this may be indicated by vibrato (i.e. a rapid wobbling up and down of the pitch) in the audio signal. The magnitude of the tremolo or vibrato effect may indicate the respective extent to which the patient's level of analgesia or paralysis has strayed from that intended or desired by the clinician.

In a typical surgical procedure, the synthesised audio signal may be output contemporaneously and in conjunction with other patient monitoring displays, such as visual displays used by clinicians to monitor patients' other physiological parameters. Furthermore, the synthesised audio signal may be output contemporaneously and in conjunction with other auditory displays, such as auditory displays providing information about the patient's pulse oximeter readings, respiration (“respiration sonification”), blood pressure (“blood pressure earcons”), as well as other auditory alarms and background noise found in medical environments. The synthesised audio signal is aurally distinguishable from the other auditory displays, so that clinicians are able to receive, distinguish and comprehend such multiple auditory displays even when presented simultaneously.

In order to increase the ease with which a clinician may distinguish the synthesised audio signal from other auditory displays and noise, the synthesised audio signal may comprise multiple pulses of sound. In a preferred embodiment, the audio signal comprises a spaced double pulse (i.e. two distinct pulses of sound). The ‘double pulse’ nature of the audio signal further assists in distinguishing the audio signal from other sounds.

In embodiments which utilise a double pulse, the second pulse preferably follows quickly after the end of the first pulse so that it is apparent that the two pulses are related and form part of the same auditory display. Whilst the pulses may not necessarily be identical to each other, the pulses are both suitably distinguishable from the “competing” auditory displays and noise.

In some embodiments which utilise an audio signal in the form of a double pulse, both pulses may be the same to ensure that the information encoded in the pulses is appreciated by the clinician. In particular, the first pulse may serve to attract the clinician's attention, and the second pulse may confirm the information conveyed in case the clinician is unable to discern that information from the first pulse.

In other embodiments which utilise an audio signal in the form of a double pulse, particularly where it is desired to provide historical or trend information regarding the patient's level of consciousness, the two pulses may be different. For example, the first pulse may represent the previous measure, while the second pulse represents the current measure. The rate of change of the patient's level of consciousness may be indicated aurally by using varying frequencies between the two pulses. For example, if the patient's level of consciousness has a higher BIS value than it did at the previous reading, then the second of the pulses may have a higher frequency (i.e. pitch) than the first pulse. Correspondingly, if the patient's level of consciousness has a lower BIS value than it did at the previous reading, then the second pulse is at a lower pitch than the first pulse. The pitch of the audio output may therefore (but need not necessarily) correlate directly with BIS value such that different frequencies may represent different BIS values or BIS value ranges.

In some embodiments, the rate at which the audio output pulses are delivered to the clinician, or their temporal spacing, may be varied in proportion to the rate of change in the patients level of consciousness.

In environments such as ambulances, emergency rooms and intensive-care units where there can be a large amount of background and other noise, the audio signal may be delivered to the clinician by way of an earpiece. This may be the same earpiece which is also used to deliver other auditory displays signal, or a separate earpiece.

In another broad form, the invention provides apparatus for indicating level of consciousness of a patient aurally, comprising

an input for receiving a measure representative of the patient's level of consciousness,

an audio synthesiser for synthesising an audio signal from the measure, and

a speaker for outputting the audio signal.

At least one audible characteristic of the audio signal is indicative of the measure of the patient's level of consciousness.

The audio synthesiser may suitably include means for varying the amplitude and/or frequency of the audio signal in accordance with detected level of analgesia or paralysis of the patient.

Typically, the audio synthesiser is part of a computer, and the audio signal is synthesised by computer software.

The apparatus of the invention may incorporate a user interface to allow the clinician to select, among other things, the desired hypnotic state BIS value range, desired levels of paralysis and/or analgesia, as well as desired volume levels, pitch levels and other user preference settings (for example, whether historical trend information is to be presented or only current hypnotic state information). The user interface is typically an electronic controller.

In order that the invention may be better understood and put into practice, a preferred embodiment thereof will now be described, by way of example only, with reference to the accompanying drawings.

### BRIEF DESCRIPTION OF THE DRAWINGS

FIGS. 1A to 1D show idealised representations of the typical changes in a patient's EEG during increasing levels of hypnosis measured with a BIS monitor;

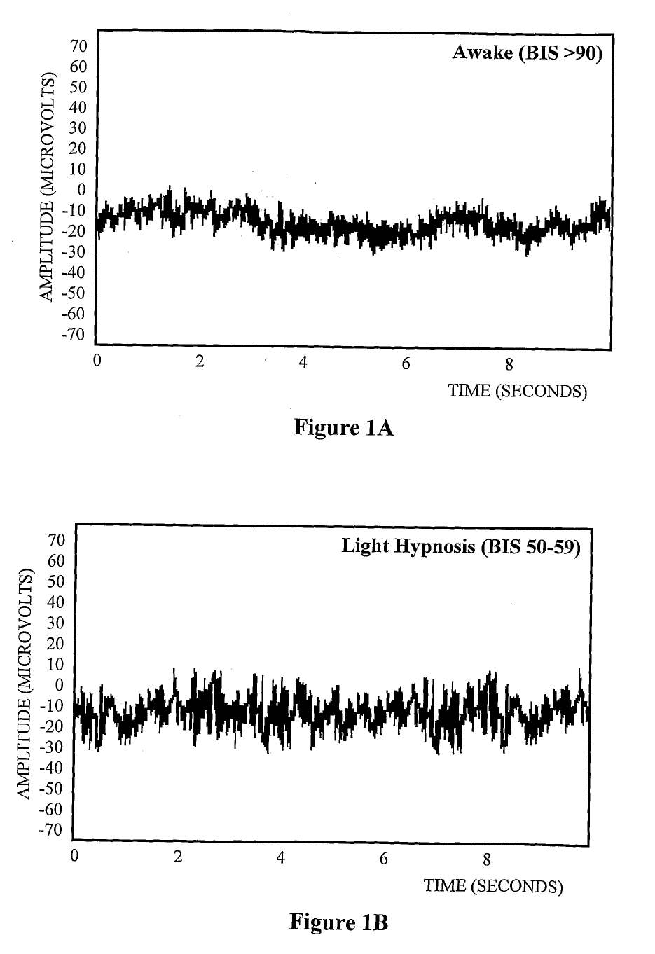

FIG. 2 contains graphical representations of certain auditory displays which may be present in a medical environment;

FIG. 3 shows a graphical representation of the frequency (i.e. pitch) and amplitude (i.e. volume) changes in the audio output produced in particularly preferred embodiments of the present invention with the variations in a patient's hypnotic state; and

FIG. 4 shows a graphical representation of the rapid amplitude variation (i.e. tremolo) in the audio output produced in particularly preferred embodiments of the present invention due to variation in the patient's level of analgesia.

### DETAILED DESCRIPTION OF EMBODIMENTS OF THE INVENTION

In the invention, a measure of the level of consciousness of a patient is represented aurally by an audio signal. In the preferred embodiment described below, the measure of the level of consciousness used is the BIS Index, and therefore the aural representation of the level of consciousness will be referred to as BIS sonification. However, it will be appreciated that other measures of the level of consciousness of a patient may be used.

The apparatus used to implement the invention is typically a computer or other signal processing device (not shown) having an input adapted to receive signals derived from EEG readings. These signals are measures of the level of consciousness of a patient. The computer or other signal processing device is adapted to synthesise audio signals dependent on the respective readings. This process is often referred to as “mapping” the received signal into the output auditory BIS sonification. Typically, the audio signals are synthesised by software. Apparatus for audio signal synthesis is known in the art, and need not be described in detail in this specification.

The audio signal is output by a speaker, which may be located on the computer or remotely, or through an earpiece.

In the preferred embodiment, the received signal is in a form representative of the patient's BIS value or range of BIS values. In other embodiments, the received signal may be in another “unprocessed” form, and the computer will perform the necessary processing to convert the received signal into a signal representative of the patient's BIS value or range of BIS values. The computer may also have a memory for storing previous received signals, to provide historical and trend information regarding the patient's level of consciousness, i.e. the patient's hypnotic state and/or level of anaesthesia. These signals may be stored as BIS values, or in some other form.

In the preferred embodiment, the audio signal is synthesised as a double pulse comprising first and second audio components. Each audio component is a relatively prolonged tone, rather than a short beep. The first component represents the next previous measure of the patient's level of consciousness, while the second component represents the current measure of the patient's level of consciousness. In this manner, the clinician can easily detect changes in the patient's level of consciousness.

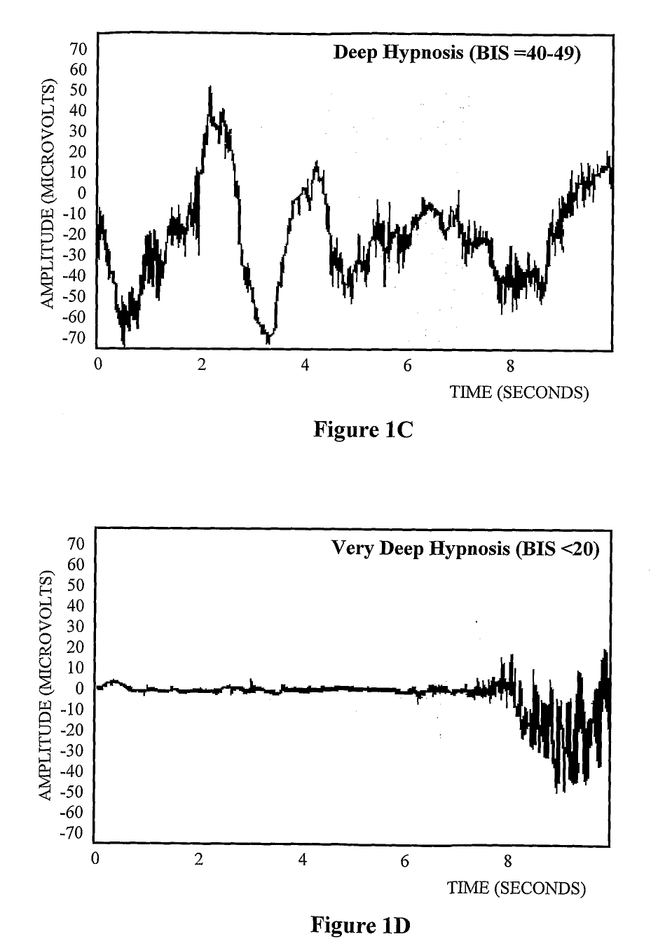

FIG. 2 shows graphical plots of the audio output amplitude profile for a number of typical auditory displays which may often be present in a medical environment. The amplitude profile of each auditory display is mapped as a function of time. FIGS. 2(a), 2(b), 2(c) and 2(d) respectively show audio signals representing pulse oximeter readings, respiratory sonification, a blood pressure earcon, and the BIS sonification of the preferred embodiment. FIG. 2 (e) shows each of the amplitude profiles represented in FIG. 2 (a)-(d) superimposed upon each other.

From FIG. 2(e) it can be seen that even if all of these displays occur at the same time, the amplitude profile of each display is sufficiently distinctive to enable the clinician to distinguish between the different displays. If the blood pressure earcon (which may often have a greater amplitude than the other displays) co-occurs with the BIS sonification, the repeated pulse of the BIS sonification helps to ensure that the BIS sonification display will still be heard by the clinician. Other differences between the BIS sonification display and the other displays, for example in terms of other sound parameters (such as pitch or timbre etc), will also help to distinguish the BIS sonification display from the other displays.

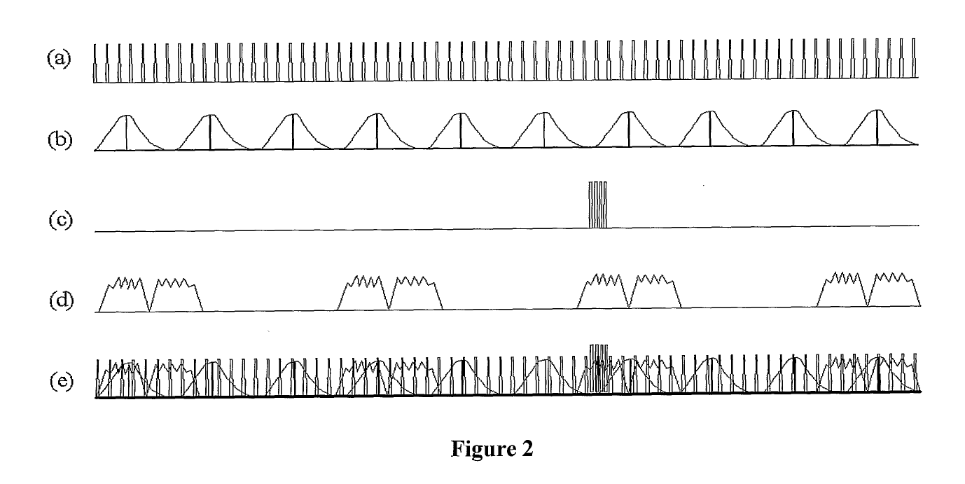

In the preferred embodiment, the audio signal is synthesised so that its amplitude (or volume) and frequency (or pitch) are proportional to the BIS value, for example as shown in FIG. 3. The frequency (or pitch) of the audio signal (shown by the solid plotted line) varies with the BIS value according to a substantially linear relationship. More specifically, at low patient BIS values (near 0), the BIS sonification will generate a relatively low (but still audibly perceptible) output frequency. As the patient's BIS value increases, the output frequency of the BIS sonification will increase correspondingly as a linear function of the patient's BIS value, to a maximum patient BIS value of 100. In other preferred embodiments, the BIS sonification may vary according to a stepwise relationship with the patient's BIS value.

The amplitude of the audio signal (shown by dashed curve in FIG. 3) varies as a nonlinear function of the patient's BIS value. This curve may approximately represent an equi-loudness curve. The minimum volume of the BIS sonification audio signal will occur when the patient's BIS value is within a desired BIS value range defined by the clinician. In the preferred embodiment, when the patient's BIS value falls within this desired range (marked “A”), the volume of the BIS sonification will be below the minimum level (Vmin) which is perceptible to the clinician, and so the BIS sonification will not be heard by the clinician. However, if the patient's BIS value strays outside the desired range, then the volume of the BIS sonification will increase above the minimum level perceptible to the clinician, so that the BIS sonification may be heard by the clinician. Typically, the volume will increase according to the extent to which the BIS value is outside the predetermined range.

The initial increase in volume when the patient's BIS value strays outside the desired range by only a small amount (in either direction) will be quite small. This is represented by the relative flatness of the dashed curve immediately on either side of the desired region. However, as the patient's BIS value strays further from the desired range, the rate of increase in volume grows significantly. This is represented by the increasingly steep portions of the dashed curve as the curve moves outwardly from the desired region. With yet further straying of the patient's BIS value, the volume of the BIS sonification may plateau or level out. In cases where the patient's BIS value strays significantly above the desired region, the levelling may correspond to the patient's decreasing state of hypnosis and early stages of consciousness. Therefore, as the patient may be regaining consciousness, it may not be necessary to further increase the volume of the BIS sonification to alert the clinician.

Conversely, when the patient's BIS value strays significantly below the desired region, the levelling may be somewhat slower than in the high BIS value case. Hence, when the patient's BIS value strays significantly below the desired region, the volume of the BIS sonification may continue to increase for longer than it does in the case where the patient's BIS value strays above the desired region, in order to provide the clinician with an increasingly urgent alert to the patient's increasing state of hypnosis. This also compensates for perceptual interaction between pitch and amplitude. Nevertheless, levelling of the volume when the patient's BIS value continues to fall may be necessary so that the BIS sonification does not become too loud and potentially inhibit the clinician's ability to monitor the other auditory displays etc.

The described embodiment attracts the attention of the clinician when the patient's level of consciousness changes in an unexpected way. When a patient is meant to be unconscious during a procedure and their level of consciousness either increases or fluctuates, the sound produced will draw the attention of the clinician to the unexpected change. In this way it acts as an information carrying alarm. It provides information as to the type of unexpected event and the magnitude of the event. In the case where the level consciousness increases at the end of the procedure as the patient awakens, the sound acts as confirmation to the clinician of the expected change in the patient state. The sonification is also innovative in that it is designed to work with existing sonifications and alarms without masking or being masked by them.

An added advantage of the preferred embodiment is that the audio signal can be modified to provide an aural indication of the patient's current level of analgesia. This may be done by modulating the amplitude of the signal to produce a tremolo effect.

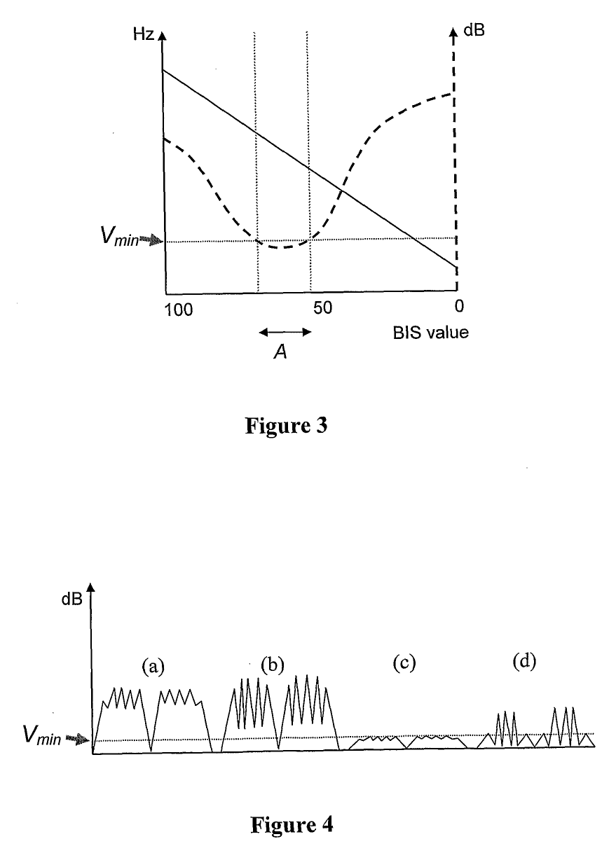

FIG. 4 provides a graphical representation of the way tremolo in the BIS sonification is used to provide an indication of the patient's current level of analgesia. FIG. 4(a) depicts a typical BIS sonification amplitude profile for a patient in a “normal” awake clinical state, and whose level of experienced pain is low (indicating a sufficient level of analgesia). For the reasons explained with reference to FIG. 3 above, the amplitude (i.e. volume) of the BIS sonification for the patient represented in FIG. 4(a) is relatively high as the patient is awake and therefore has a relatively high BIS value. Also, the amount of tremolo (i.e. rapid variation in volume) displayed in FIG. 4(a) is low because the patient's level of experienced pain is low.

In contrast, FIG. 4(b) depicts a typical BIS sonification amplitude profile for a patient in an awake clinical state, but whose level of experienced pain is high (indicating an insufficient level of analgesia). The patient's level of experienced pain is recognisable from the BIS sonification by the relatively high amount of tremolo displayed.

FIG. 4(c) depicts a BIS sonification amplitude profile for a patient whose hypnotic state or level of consciousness is within the desired range defined by the clinician, and whose level of analgesia is sufficient. This is represented by the amplitude of the BIS sonification being below the minimum level audible by the clinician, and the level of tremolo being low.

Finally, FIG. 4(d) depicts a BIS sonification amplitude profile for a patient whose hypnotic state is within the desired range, but whose level of analgesia is insufficient. This is represented by the amplitude of the BIS sonification being generally below the minimum level audible by the clinician, but the large amount of tremolo (indicating experienced pain) causes the BIS sonification to “waver” into the clinician's audible range.

In a similar manner, the pitch of the audio signal can be modified to provide vibrato as an aural indication of the patient's current level of paralysis.

It is to be understood that the terminology employed above is for the purpose of description and should not be regarded as limiting.

The foregoing embodiment is intended to be illustrative of the invention, without limiting the scope thereof. The invention is capable of being practised with various modifications and additions as will readily occur to those skilled in the art. For example, the level of analgesia or paralysis of the patient may be sonified independently of the level of consciousness.

Accordingly, it is to be understood that the scope of the invention is not to be limited to the exact operation described and illustrated, but only by the following claims which are intended, where the applicable law permits, to include all suitable modifications and equivalents within the spirit and concept of the invention.

Throughout this specification, including the claims, where the context permits, the term “comprise” and variants thereof such as “comprises” or “comprising” are to be interpreted as including the stated integer or integers without necessarily excluding any other integers.

What is claimed is:

1. A method of indicating level of consciousness of a patient aurally, comprising the steps of:
(a) obtaining a measure representative of the patient's level of consciousness,
(b) synthesizing an audio signal from the measure,
(c) outputting the audio signal,
wherein at least one audible characteristic of the audio signal is dependent upon the patient's level of consciousness; and
(d) repeating steps (a) to (c) at intervals of time.

2. A method as claimed in claim 1, wherein the measure representative of the patient's level of consciousness is a BIS value.

3. A method as claimed in claim 2, wherein the pitch of the audio signal is dependent on the BIS value.

4. A method as claimed in claim 2, wherein the amplitude of the audio signal is dependent on the BIS value.

5. A method as claimed in claim 4 wherein the amplitude of the audio signal is low if the BIS value is within a predetermined range, but audibly increases according to the extent to which the BIS value is outside the predetermined range.

6. A method as claimed in claim 1, wherein the audio signal comprises first and second audio components spaced in time.

7. A method as claimed in claim 6, wherein the second audio component is an audio signal synthesized from the current measure, and the first audio component is an audio signal synthesized from the previous measure.

8. A method as claimed in claim 1, further including the steps of:
(a) detecting level of paralysis of the patient, and
(b) modifying the audio signal according to the detected level of paralysis,
whereby at least one audible characteristic of the modified audio signal is dependent upon the patient's paralysis.

9. A method as claimed in claim 1, further including the steps of:
(a) detecting level of analgesia of the patient, and
(b) modifying the audio signal according to the detected level of analgesia,
wherein at least one audible characteristic of the modified audio signal is dependent upon the patient's analgesia.

10. A method as claimed in claim 8, when wherein the step of modifying the audio signal comprises modulation of the amplitude and/or frequency of the audio signal.

11. A method as claimed in claim 1 wherein the audio signal is output as one of a plurality of audio signals representing other physiological parameters of the patient, and wherein the audio signal is aurally distinguishable from the other one(s) of the plurality of audio signals.

12. A method as claimed in claim 1 wherein the audio signal is output via an earpiece.

13. A method of representing level of consciousness of a patient aurally, comprising the steps of:
(a) obtaining measures representative of the patient's level of consciousness at spaced intervals of time,
(b) synthesizing an audio signal having at least first and second audio components, the second audio component being dependent on a current measure, and the first audio component being dependent on the next previous measure, whereby at least one audible characteristic of each component of the audio signal is indicative of the respective measure, and
(c) outputting the audio signal.

14. A method as claimed in claim 13, further comprising the step of varying the amplitude and/or frequency of the audio signal in accordance with detected level of paralysis or analgesia of the patient.

15. Apparatus for indicating level of consciousness of a patient aurally, comprising:
an input for receiving a measure representative of the patient's level of consciousness,
an audio synthesizer for synthesizing an audio signal from the measure, and
a speaker for outputting the audio signal,
wherein at least one audible characteristic of the audio signal is indicative of the measure of the patient's level of consciousness.

16. Apparatus as claimed in claim 15, wherein the audio synthesizer includes means for varying the amplitude and/or frequency of the audio signal in accordance with detected level of paralysis or analgesia of the patient.

17. Apparatus as claimed in claim 15, wherein the audio synthesizer is part of a computer, and the audio signal is synthesized by computer software.

18. Apparatus as claimed in claim 15, wherein the speaker is part of an earpiece.

19. A method as claimed in claim 3, wherein the amplitude of the audio signal is dependent on the BIS value.

20. A method as claimed in claim 9, wherein the step of modifying the audio signal comprises modulation of the amplitude and/or frequency of the audio signal.
